(19) United States
(12) Patent Application Publication
(10) Pub. No.: US 2025/0186311 A1
(43) Pub. Date: Jun. 12, 2025

(54) ARTICLES FOR STREAMLINED SKIN REJUVENATION AND WOUND HEALING USING EXOSOMES AND PRP

(71) Applicants: Reza Mollaaghababa, Natick, MA (US); Setareh Sharif, Natick, MA (US); Ramin M. Hakami, Natick, MA (US); Mehdi Abedi, Natick, MA (US)

(72) Inventors: Reza Mollaaghababa, Natick, MA (US); Setareh Sharif, Natick, MA (US); Ramin M. Hakami, Natick, MA (US); Mehdi Abedi, Natick, MA (US)

(21) Appl. No.: 18/776,599

(22) Filed: Jul. 18, 2024

Related U.S. Application Data
(60) Provisional application No. 63/527,497, filed on Jul. 18, 2023.

(51) Int. Cl. A61K 8/02; A61K 8/14; A61K 8/98

(52) U.S. Cl. CPC A61K 8/0212; A61K 8/14; A61K 8/985

(57) ABSTRACT

In one aspect, a facial mask is disclosed, which comprises a polymeric matrix having a plurality of protrusions configured for penetrating into the stratum corneum of facial skin, and a plurality of exosomes that is distributed within the polymeric matrix, where the polymeric matrix is degradable when embedded within the skin and/or the physiological temperatures, e.g., 37° C. or higher, so as to release the exosomes into the skin.

### RELATED APPLICATION

The present application claims priority to provisional application No. 63/527,497 titled “Articles for Streamlined Skin Rejuvenation and Wound Healing Using Exosomes and PRP,” which was filed on Jul. 18, 2023, and which is herein incorporated by reference in its entirety.

### BACKGROUND

The present teachings are generally directed to dermal patches, face masks, and wound dressings that include a polymeric matrix in which a plurality of exosomes and/or platelet rich plasma (PRP) are embedded, where the polymeric matrix is degradable in the skin to release the exosomes and/or PRPs.

A number of techniques are known for skin rejuvenation. Some of the skin rejuvenation techniques employ various mechanisms of causing tissue damage to promote the regeneration of collagen and other skin components. Further, a variety of wound dressings are known in the art. As the number of diabetic and elderly patients increases, wound dressings with enhanced wound healing capabilities are increasingly in demand.

### SUMMARY

In one aspect, a facial mask is disclosed, which comprises a polymeric matrix having a plurality of protrusions configured for penetrating into the stratum corneum of facial skin, and a plurality of exosomes that is distributed within the polymeric matrix, where the polymeric matrix is degradable when embedded within the skin and/or the physiological temperatures, e.g., 37° C. or higher, so as to release the exosomes into the skin.

In some embodiments, the protrusions are shaped and sized so as to allow penetration thereof at least partially into the dermal layer. By way of example, the protrusions can have a height in a range of about 100 micrometers (microns) to about 800 microns, e.g., in a range of about 200 microns to about 600 microns. In some embodiments, the number of exosomes contained in the polymeric matrix layer can be in the range of about 2 billion to about 4 billion, e.g., 3 billion. Some examples of suitable materials that can be employed for forming the polymeric matrix include any of hydrogel, chitosan, chitin, silk, carboxymethyl cellulose (CMC), chondroitin, collagen, and gelatin.

Further, in some embodiments, the facial mask can have a thickness in a range of about 0 5 mm to about 5 mm, e.g., 2 mm, though other thicknesses can also be employed

The facial mask can also include a polymeric backing layer to which the exosome- and/or PRP-loaded polymeric matrix can be bonded.

In some embodiments, the facial mask can include an adhesive border for securing the mask to a subject's face.

In some embodiments, in addition to or instead of the exosomes, a facial mask according to the present teachings can incorporate one or more anti-aging and/or skin rejuvenating agents within the polymeric matrix (e.g., within the protrusions) so as to release these agents into a subject's dermal layer. Such agents may include, for example, antioxidants, growth factors (GF), cell regulators, among others, which can have direct effects on collagen metabolism and/or influence collagen production.

Some examples of antioxidants that can be incorporated into the above mask include, without limitation, vitamins C, B₃, and E. By way of example, vitamin C (L-ascorbic acid) in a concentration between 5 and 15% can be incorporated in the mask's polymeric matrix (e.g., in the projections). Without being limited to any particular theory, it is expected that the release of the vitamin C into skin can lead to anti-aging effect, e.g., inducing the production of collagent type 1 and collagent type 3 as well as enzymes that can facilitate the production of collagen, and inhibitors of matrixmetalloproteinase.

In some embodiments, vitamin E (α-tocopherol) may be incorporated in the polymeric matrix of a facial mask according to the present teachings, e g., in a range of about 2% and about 20%. In some embodiments, a combination of vitamin C and vitamin E can be incorporated in the polymeric matrix of the mask. In yet other embodiments, vitamin B₃ (Niacinamide) can be incorporated, e.g., in a concentration of about 5%. In yet another embodiment, vitamin A (retinol) and/or its derivatives (retinaldehyde and tretinoin), which have antioxidant effects, may be incorporated in the polymeric matrix of the facial mask, e.g., in a concentration in of about 0.05%. Without being limited to any particular theory, the introduction of the vitamin A and/or its derivative can induce the biosynthesis of collagen and can reduce the expression of MMP 1 (collagenase 1).

In yet another embodiment, a facial mask according to the present teachings can include hyaluronic acid (HA) incorporated within its polymeric matrix. In some embodiments, the polymeric matrix of the mask may include only hyaluronic acid while in other embodiments a combination of hyaluronic acid and any of the above anti-aging and/or skin rejuvenating agents and/or exosomes may be incorporated in the polymeric matrix of the facial mask

Further, in some embodiments, a facial mask according to the present teachings in which hyaluronic acid and/or any of the above anti-aging and/or skin rejuvenating agents are incorporated can lack the above protrusions and rather include a substantially flat surface. In some such embodiments, the contact of the mask surface with the skin can result in a slow release of these agents onto the subject's skin.

In a related aspect, a wound dressing is disclosed, which includes a polymeric matrix layer in which a plurality of exosomes and/or PRPs is distributed. In addition, or alternatively, the polymeric matrix can include one or more reservoirs (which are also herein referred to as the micro reservoirs, though the reservoirs are not necessarily limited to those having sizes in the micron range) in which a plurality of exosomes and/or PRPs are stored. A substrate is bonded to the polymeric matrix and is configured for passage of wound exudates therethrough. The wound dressing can further include a polymeric base layer to which the substrate is bonded. The polymeric matrix and the polymeric base layer can be formed, for example, of one or more polymers disclosed herein. As discussed in more detail below, in some embodiments, the substrate can be in the form of cellulose acetate gauze coated with a hydrophobic, tacky, crosslinked silicone gel

In one aspect, a cosmetic composition for inducing dermal fibroblast proliferation and differentiation in adult skin is provided. Such a cosmetic composition may include exosomes derived from human foreskin fibroblasts, or from embryonic fibroblast cell lines. The cosmetic composition may further include differentiation-inducing factors such as miRNAs, various peptides, growth factors and the like, extracted from exosomes secreted by the human foreskin fibroblasts, or embryonic fibroblast cells The cosmetic composition may further include a human foreskin fibroblast or embryonic cell lines culture medium, but not limited thereto. In some embodiments, the cosmetic composition can be incorporated a facial mask and/or a wound dressing such as those described above and further discussed below. By way of example, a facial mask containing such a cosmetic composition may be formed of a polymeric material, such as those discussed herein, providing a polymeric matrix through which the cosmetic composition can be distributed. In some embodiments, such a facial mask may include a plurality of protrusions that can penetrate the skin's stratum corneum to be placed at least partially within the skin. Once within the skin, the polymeric protrusion can degrade to release the cosmetic composition into the skin, e.g., into the epidermal or more typically the dermal layer. In other embodiments, a facial mask formed of a polymer may have a substantially flat skin-contacting surface. The skin-contacting surface of such a facial mask, which incorporates the cosmetic composition, can be placed against the skin to allow a slow release of the cosmetic composition onto the skin surface with some penetrating the skin.

Further, in some embodiments, the cosmetic composition can be incorporated into a wound dressing.

Further understanding of various aspects of the present teachings can be obtained by reference to the following detailed description in conjunction with the associated drawings, which are described briefly below.

### BRIEF DESCRIPTION OF THE DRAWINGS

FIG. 1A schematically depicts a schematic perspective view of a facial mask in accordance with the present teachings.

FIG. 1B schematically depicts a cross-sectional view of the facial mask depicted in FIG. 1A.

FIG. 1C is a partial schematic view of FIG. 1A, illustrating one of the protrusions of the facial mask of FIG. 1A.

FIG. 1D schematically depicts an individual wearing the facial mask of FIG. 1A.

FIG. 2 is a partial schematic cross-sectional view of another embodiment of a facial mask according to the present teachings.

FIG. 3A schematically depicts various layers of the skin.

FIG. 3B schematically depicts the penetration of the protrusions of the facial mask depicted in FIG. 1A into the skin.

FIG. 4A is a cross-sectional view of a dermal patch according to an embodiment of the present teachings.

FIG. 4B is a top schematic view of the dermal patch depicted in FIG. 4A.

FIG. 5A is a cross-sectional view of a wound dressing according to an embodiment of the present teachings.

FIG. 5B is a perspective view of the exosome- and/or PRP-loaded polymeric matrix and a substrate, through which wound exudate can pass, of the wound dressing of FIG. 5A.

FIG. 5C is a top schematic view of the wound dressing of FIG. 5A, showing an adhesive border that allows retaining the wound dressing in contact with a patient's wound.

FIG. 6 is a schematic cross-sectional view of a wound dressing according to another embodiment of the present teachings.

### DETAILED DESCRIPTION

The following detailed description refers to the accompanying drawings. The same or similar reference numbers may be used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The present disclosure is generally related to dermal patches, face masks, and wound dressings for delivery of exosomes and/or PRP to skin for skin rejuvenation, and wound healing, among others. Further, the present disclosure is directed to wound dressings that incorporate exosomes and/or PRP, e.g., via distribution within a polymeric matrix, for use in skin graft applications

As discussed in more detail below, such a dermal patch, face mask or wound dressing can include a polymeric matrix in which a plurality of exosomes and/or PRP is dispersed As discussed in more detail below, upon application of the dermal patch, face mask or dressing to a subject's skin or a wound, the polymeric matrix can release the exosomes and/or PRP over a time period into the skin, or the wound, to effectuate a desired result, e.g., skin rejuvenation and/or wound healing.

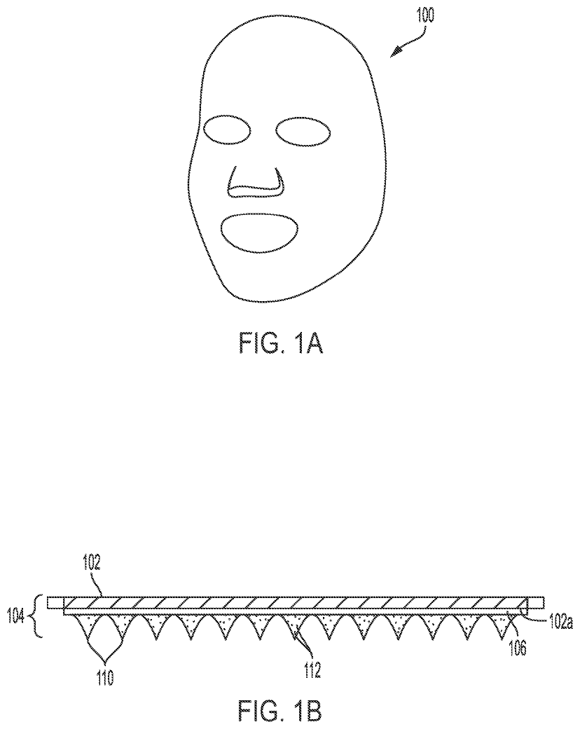

With reference to FIGS. 1A, 1B, 1C. ID, a face mask 100 according to an embodiment of the present teachings includes a flexible polymeric backing 102 that allows conforming the face mask to a subject's face and a polymeric matrix 104 that is bonded to an inner surface 102a of the polymeric backing 102. The coupling of the polymeric matrix 104 to the backing 102 can be achieved using a variety of techniques. By way of example, in some embodiments, the polymeric backing 102 can be glued to the inner surface of the polymeric backing 102a.

In some embodiments, the polymeric backing 102 can be formed of a biocompatible polyester polymer and copolymer, such as, polylactic acid (PLA), polyglycolic acid (PGA), poly-lactide-co-glycolide (PLGA) and polydioxanone (PDS) or derivatives thereof, though any suitable polymeric material can be employed.

The polymeric matrix 104 includes a base portion 106 from which a plurality of projections 110 (herein also referred to as “shafts” or “microneedles”) extend. As discussed in more detail below, in many embodiments, the projections 110 are configured for penetration through the stratum corneum and the epidermis to be introduced at least partially into the dermal layer of the skin. By way of example, in some embodiments, the projections 110 can have a height (H) in a range of about 100 microns to about 800 microns, or 200 microns to about 400 microns and a base diameter (BD) in a range of about 300 microns to about 500 microns, e.g., 400 microns.

In this embodiment, the protrusions have a conical shape extending from a base to a tip, though other shapes, such as pyramidal, cylindrical, or other suitable shapes can also be employed. The draft angle (o) of the protrusions is selected to allow their facile penetration into the stratum corneum layer of the skin. By way of example the angel (o) can be in a range of about 5 degrees to about 10 degrees, though other values may also be employed.

In some embodiments, the base portion 106 and the protrusions 110 are formed separately and subsequently joined together using, e.g., glue. In other embodiments, the base portion 106 and the protrusions 110 can be formed as an integral unit. Further, while in some embodiments, the base layer and the projections are formed of the same polymer, in other embodiments, different polymers may be employed for the formation of the base layer and the projections.

With continued reference to FIG. 1A, a plurality of exosomes 112 is distributed in the polymeric matrix and particularly in the projections 110 In some embodiments, the exosomes are incorporated only in the protrusions and not in the base layer. As discussed further below, in some embodiments, the exosomes 112 can be derived from stem cells. By way of example, the exosomes can be collected from a variety of different types of cultured stem cells, such as mesenchyme stem cells derived from bone marrow, adipose tissue, including stem cells derived from a young subject (e.g., less than 20 years of age); or an embryo, a fetus (including umbilical cord and/or placenta) or from a mixture of such sources; fibroblasts and induced pluripotent stem cells (IPSCs).

An example of a process that can be employed for collecting such exosomes is described in the Example section below.

In this embodiment, the polymeric matrix 104 is formed of a biodegradable material that will degrade and dissolve when inserted into the skin, thereby releasing the exosome cargo into the skin, e.g., into the dermal layer The rate of release of the exosomes into the skin can be varied by using different biodegradable polymers exhibiting different dissolution rates in the skin. For example, in some embodiments, the dissolution rate of the polymeric material forming the projections is such that at least about 80%, or at least about 90%, or 100% of the exosomes incorporated in the projections are released within a temporal period in a range of about 1 to about 2 hours, though the polymeric material can also be selected to exhibit other dissolution rates

Some examples of suitable biodegradable polymeric materials include, without limitation, hydrogel, chitosan, chitin, silk, carboxymethyl cellulose (CMC), chondroitin, collagen, and gelatin, among others.

In this embodiment, an adhesive strip 120 is formed around the outer edge of the inner surface of the mask to allow the retention of the mask on the subject's skin Some examples of suitable adhesives include, without limitation, a woven fabric, plastic (PVC, polyethylene, or polyurethane) or a latex strip. In other embodiments, the mask may not include an outer adhesive strip.

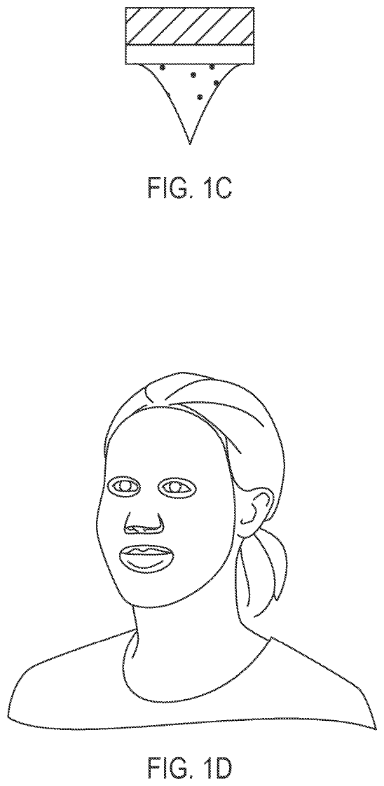

FIG. 2 schematically depicts another embodiment of a mask 200 according to the present teachings, which has a similar structure as the mask 100, including a polymeric matrix 202 in which a plurality of exosomes and/or PRP 202a is distributed. In addition, in this embodiment, the polymeric matrix is loaded with a quantity of platelet rich plasma (PRP) 202b. As known in the art. PRP is a concentrate of platelet-rich plasma protein that is derived from whole blood and is centrifuged to remove red and white blood cells and clotting factors.

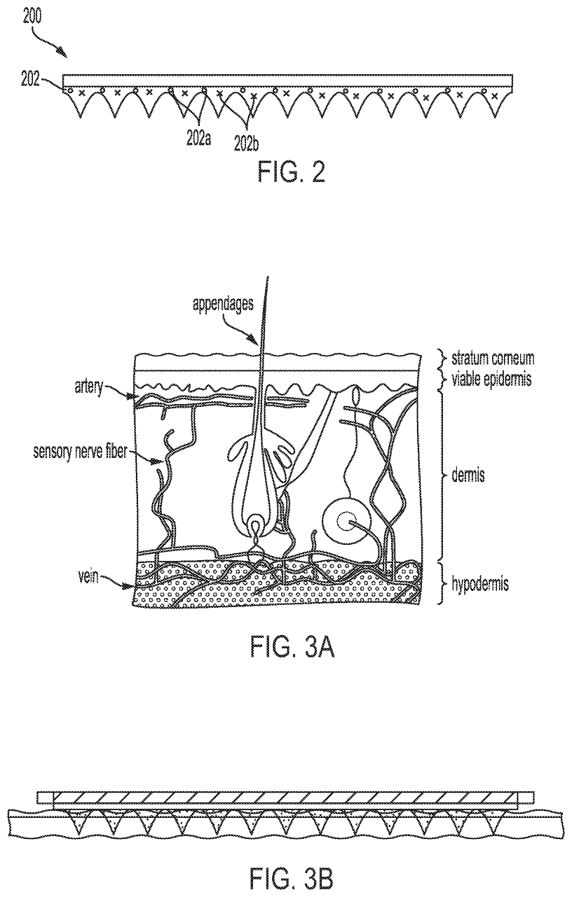

FIG. 3A schematically depicts various layers of the skin, which include the stratum corneum, the epidermis, the dermis and the hypodermis. As discussed in more detail below, in some embodiments the height of the projections is selected to ensure that upon application of the patch to a subject's skin the protrusions penetrate at least partially into the dermal layer. Without being limited to any particular theory, it is expected that the release of the exosomes into the dermal layer will stimulate the fibroblast cells residing in this layer to produce collagen and hence help rejuvenate the skin.

More specifically, as shown schematically in FIG. 3B, upon placement of the mask 100 or 200 on a subject's face, the protrusions can penetrate through the stratum corneum and the epidermal layer to be introduced at least partially into the dermal layer. The polymeric matrix will then begin to dissolve, thereby releasing at least a portion of the exosomes and/or RPRs into the dermal layer. As noted above, the introduction of the exosomes into the dermal layer is expected to stimulate collagen production via stimulation of the fibroblasts residing in this layer.

In some embodiments, the exosomes incorporated in the polymeric matrix are in the form of lyophilized particles, which are reconstituted upon introduction into the dermal layer Exosomes are key elements of cell-to-cell communication and orchestration of tissue repair and regeneration in response to injury in adults. When applied to a partially “injured” epidermal/dermal layer, they regulate repair by way of paracrine signaling between cells, without the need for direct contact between them.

Exosomes contain molecules that can stimulate proliferation and differentiation of resident stem cells and recruitment of other stem cells to the area to initiate regeneration. Exosomes carry signaling molecules in the form of peptides, DNA, microRNA, non-coding RNAs, cytokines, lipids, and the like.

In some embodiments of the masks 100 and 200, the projections are releasably coupled to the base of the polymeric matrix such that upon the penetration of the projections into the dermal layer, the projections are separated from the base portion of the polymeric matrix to be lodged into the dermal layer. Once lodged in the dermal layer, the polymeric matrix of the polymeric projections will dissolve to release the exosomes and/or PRP incorporated therein into the dermal layer.

In some embodiments, a face mask according to the present teachings can contain about 2 to about 4 billion exosomes therein In some embodiments, the concentration of the platelets in the PRP can be about 2.5 to about 9 times the concentration of the platelets found in normal blood (the average blood platelet count is in a range of about 300,000/microliter of 300M per ml). By way of example, the concentration of the platelets in PRP can be at least about 1,000,000/mL, e.g., about 1,500,000,000/ml.

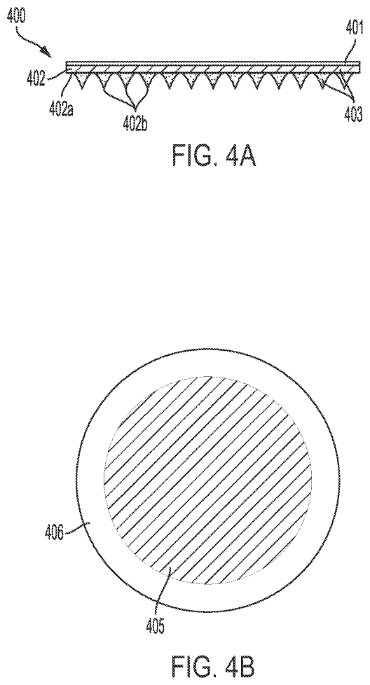

In some embodiments, a dermal patch can be used for ameliorating and treating male pattern baldness. By way of example, FIGS. 4A and 4B schematically depict such a dermal patch 400 that includes a polymeric backing 401 and a layer of a polymeric matrix 402 that is bonded to the polymeric backing, e.g., glued to the polymeric backing. The polymeric matrix layer 402 includes a base layer 402a from which a plurality of projections 402b extend, where the projections are configured for penetration through the stratum corneum and the epidermis. A cargo of exosomes and/or PRP 403 is distributed within the polymeric matrix layer 402 and particularly within the projections 402b. In some embodiments, the exosomes and/or PRP is primarily, or solely, distributed within the projections. A release layer 405 can cover the corrugated surface of the polymeric matrix layer 402 and a transparent adhesive layer 406 can cover the external surface of the base layer to facilitate placing the wound dressing on a portion of a subject's scalp and securing it in place.

It is expected that the released exosomes/PRP will activate the stem cells residing in dermal and/or follicular tissue to help with hair growth. In some implementations of this embodiment, the height of the projections 403 can be in a range of about 100 to about 500 microns, e g., in a range of about 200 to about 300 microns. Further, in some embodiments, the projections can be releasably coupled to the base layer of the polymeric matrix so that they can be released into the skin layer into which the projections penetrate. This allows removing the dermal patch after a certain period.

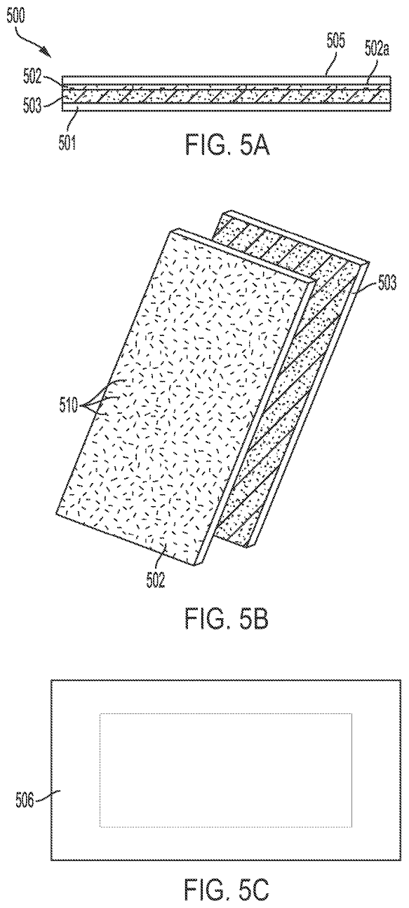

With reference to FIG. 5, in a related aspect, the present teachings provide a wound dressing 500 that includes a polymeric matrix layer 502 in which a plurality of exosomes and/or PRP 502a is incorporated More specifically, the wound dressing 500 includes a base layer 501, which can be formed of a variety of suitable polymeric materials. By way of example, the base layer 501 can be in the form of a semipermeable microporous polyurethane backing layer. In some embodiments, the base layer 501 may be coated with a polyurethane pressure-sensitive adhesive layer.

The wound dressing 500 can further include a siliconized substrate 503. In some embodiments, the siliconized substrate 503 includes cellulose acetate gauze coated with a hydrophobic, tacky, crosslinked silicone gel The silicone composition penetrates the gauze to form a single, chemically homogeneous silicon phase coating the strands of the gauze. The siliconized substrate 503 can include a plurality of apertures to allow passage of wound exudates therethrough. In some embodiments, the cellulose acetate gauze can have a density of about 107 grams per square meter.

The exosome/PRP-loaded polymeric matrix layer 502 is covered by a release sheet 505 formed, for example, of siliconized paper that can be removed to expose the polymeric matrix layer.

In use, after the removal of the release sheet 505, the exosome/PRP loaded polymeric matrix is placed in contact with a wound. As the polymeric matrix is dissolved, the exosomes/PRP are released. After complete dissolution of the polymeric matrix, the siliconized substrate 503 will be in contact with the wound.

In some embodiments, a transparent adhesive layer 506 can be placed on the outer surface of the base layer to facilitate maintaining the wound dressing in place.

Further information regarding the siliconized substrate 503, as well as the base and the release layers can be obtained in U.S. Pat. No. 10,568,767, which is herein incorporated by reference in its entirety

In some embodiments, the polymeric matrix layer 502 can have an area in a range of about 1 cm² to about 100 cm² and a thickness in a range of about 0.5 mm to about 2 mm, though other thicknesses and/or surface areas can also be employed. In some embodiments, the wound dressing 500 can contain about 20 to about 40 billion exosomes.

The use of a exosome/PRP loaded polymeric matrix in a wound dressing can facilitate its healing. This can be particularly advantages in healing diabetic wounds.

In some embodiments, the wound dressing 500 can also include one or more wicking layers.

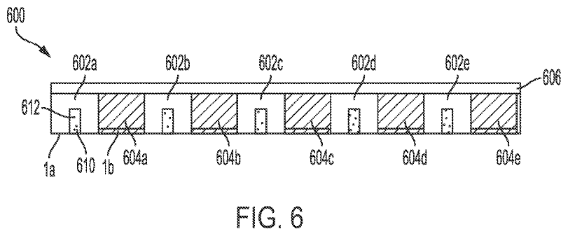

FIG. 6 schematically depicts a wound dressing 600 according to another embodiment, which includes a plurality of polymeric matrix layers 602a, 602b, 602c, 602d, and 602e (herein collectively referred to as polymeric matrix layers 602) in which a plurality of exosomes and/or PRP is incorporated. A plurality of layers 604a, 604b, 604c, 604d, and 604e (herein collectively referred to as layers 604) that provide openings through which wound exudate can pass are interposed between the polymeric matrix layers 602. In this embodiment, the layers 602 and 604 are implemented as a plurality of concentric rings. In other embodiments, other arrangements of these layers relative to one another can be employed.

The polymeric matrix layer 602 as well as the layers 604 are bonded to a polymeric backing layer 606.

The polymeric matrix layer 602 can be formed from one of the polymeric materials discussed above. Similar to the previous embodiment, the layers 604 can include cellulose acetate gauze coated with a hydrophobic, tacky, crosslinked silicone gel, or other suitable polymeric materials known in the art for use in wound dressings.

The polymeric matrix layers 602 and the exudate-receiving layers 604 include bottom portions, such as bottom portions 1a and 1b, which provide skin-contacting surfaces. Further, a polymeric release layer similar to that described above in connection with the previous embodiment (not shown here) can cover the skin-contacting surface of the polymeric matrix layers 602 and the exudate-receiving layers 604, which in use can be removed to expose the skin-contacting surface of these layers for being placed in contact with a skin wound.

In this embodiment, each of the polymeric matrix layers includes a central reservoir, such as cavity 610, associated with the polymeric matrix layer 602a. A plurality of exosomes/PRP can be stored within the central cavity of at least some of the polymeric matrix layers. In some embodiments, the total number of exosomes stored in the cavities can be, for example, in a range of about 20 billion to about 40 billion.

In some embodiments, each of the polymeric matrix layers is formed of a biodegradable polymer such that at least the bottom portion of each layer will dissolve in response to contact with a skin wound to release the exosomes and/or PRPs stored in the cavities associated with the polymeric matrix layers into the wound. The wound dressing can remain in contact with the wound.

When placed in contact with a wound, the bottom portions of the polymeric matrix layers can be dissolved (over a temporal period, which can be varied, for example, by adjusting the thickness of the bottom portions) so as to release the exosomes/PRP stored in the cavities.

In some embodiments, the bottom portions of the different polymeric matrix layers can be configured so as to release the exosomes and/or PRPs over different time scales. By way of example, in some such embodiments, the release time of the exosomes and/or PRPs can be controlled by adjusting the thickness of the bottom portions. In this manner, the temporal period over which the exosomes and/or PRPs are released can be extended.

In some embodiments, the bottom portion of each polymeric matrix layer can be formed of a different polymer and the remainder of the polymeric matrix layer. For example, in some embodiments, the bottom layer can be formed of a biocompatible polymer that is degraded in response to exposure to a patient's physiological temperature.

In some embodiments, in addition to or instead of storing the exosomes/PRP in the cavities associated with polymeric matrix levels, the exosomes/PRP can be distributed within at least a portion of the polymeric matrix surrounding the cavity. Upon dissolution of the bottom portions of the cavities, the polymeric matrix portion surrounding the cavity can be dissolved over a time period to gradually release the exosomes/PRP into the wound. In some such embodiments, the exosomes/PRP stored in the cavities can be quickly released into the wound upon the dissolution of the bottom portions of the polymeric matrix layers, and the exosomes/PRP incorporated in the matrix portions surrounding the cavities can be released over an extended time period. While in the above embodiments, the cavities are depicted as partially extending through the polymeric matrix layers, in other embodiments, they can extend further to the polymeric backing layer or in close proximity thereof.

In this embodiment, the bottom portions of the exudate-receiving layers 604 can include an adhesive layer (e.g., silicone), which contains a plurality of openings through which the exudate can pass. Further, in some embodiments, the wound dressing can include one or more wicking layer to facilitate the removal of wound exudates.

In another aspect, the present teachings relate to a dermal patch that can facilitate the treatment of acne scars. By way of example, such a dermal patch can have a configuration similar to that of the patch depicted in FIG. 4A, with or without the projections, where a plurality of adipose tissue stem cell-derived exosomes is incorporated in the polymeric matrix of the patch. In some embodiments, such a mask may be utilized as an adjuvant therapy after the application of CO2 laser treatment (e.g., fractional CO2 laser treatment). Alternatively, such a mask may be utilized as the primary treatment modality for acne scars.

In addition to or instead of adipose tissue stem cell-derived exosomes, a mask according to the present teachings for treatment of acne scars may also incorporate other ingredients. Some examples of such ingredients include, without limitation, retinoids, alpha and beta hydroxy acids, azaleic acid, niacinamide, vitamins C and E, hydroquinone, kojic acid, resorcinol and any combination of two or more of these ingredients.

One embodiment of the present invention provides a cosmetic composition for inducing dermal fibroblast proliferation and differentiation in adult skin. A cosmetic composition may include exosomes derived from human foreskin fibroblasts, or from embryonic fibroblast cell lines. The cosmetic composition may further include differentiation-inducing factors such as miRNAs, various peptides, growth factors and the like, extracted from exosome secreted by the said human foreskin fibroblasts, or embryonic fibroblast cells. The cosmetic composition may further include a human foreskin fibroblast or embryonic cell lines culture medium, but not limited thereto.

In some embodiments, a facial mask or a wound dressing according to the present teachings can incorporate the cosmetic composition. More specifically, the cosmetic composition can be distributed within the polymeric matrix of the facial masks and/or wound dressings described above. While in some embodiments, such a facial mask can include a plurality of protrusions, in other embodiments, the facial mask can have a substantially flat skin-contacting surface.

In all of the above embodiments, in some implementations, the exosomes are incorporated in the polymeric matrix and/or stored in a cavity in a lyophilized form (e.g., as a powder)

### EXAMPLE

Exosomes can be isolated from culture media of stem cells. For example, cells can be counted and plate in T-12 flasks or 35 mm culture plates in DMEM (Dulbecco's Minimum Essential Medium), supplemented with 20% FBS (Fetal Bovine Serum) and 1% antibiotic-antimycotic. The cell culture can be incubated at 37° C. and 5% CO₂ for 48 hours. The cells can be fed with fresh medium every 48 hours until they reach 90-95% confluence, typically in 2-3 weeks. Approximately 48 hours prior to harvesting of the exosomes, cells can be treated with serum-free media to exclude the presence of any serum-derived exosomes.

Exosomes can be isolated from the culture media by filtering the media through a filter having 0.22 micron pore size. The exosomes can then be extracted from the filtered media using a commercially available exosome isolation kit, such as the Genexosome Technologies GET™ Exosome isolation kit (Ca #: GET301-10). The extracted exosomes can be kept at a temperature of −80° C. until used. To check for the presence of exosomes, western blot analysis of exosome protein extracts, or transmission electron microscopy can be used. To measure exosomes quantity, Nanoparticle Tracking machines such as NanoSight NS300 can be used.

For fabrication of a dermal patch, a mold can be formed with cavities as the negative template of the projections of the dermal patch. A plurality of exosomes (e.g., in the formed of a powder) can be mixed within a quantity of a molten polymer and the mixture of the polymer and the exosomes can be poured into the mold and allowed to be hardened so as to form the dermal patch.

Those having ordinary skill in the art will appreciate that various changes can be made to the above embodiments without departing from the scope of the present teachings.

1. A facial mask, comprising:
a polymeric matrix having a plurality of protrusions configured for penetrating into stratum corneum of facial skin, and
a plurality of exosomes distributed within said polymeric matrix, wherein said polymeric matrix is degradable when embedded within the skin so as to release said exosomes into the skin.

2. The facial mask of claim 1, wherein said protrusions are shaped and sized so as to allow penetration thereof at least partially in the epidermal layer.

3. The facial mask of claim 1, wherein said protrusions have a height in a range of about 200 nm to about 800 nm.

4. The facial mask of claim 1, wherein a number of the exosomes contained in said facial mask is in a range of about 2 billion to about 4 billion.

5. The facial mask of claim 1, wherein said facial mask has a thickness in a range of about 0.5 mm to about 2 mm.

6. The facial mask of claim 1, wherein said polymeric matrix layer comprises any of.

7. The facial mask of claim 1, further comprising a polymeric backing layer to which said polymeric matrix layer is bonded.

8. The facial mask of claim 1, wherein said polymeric matrix comprises any of hydrogel, chitosan, chitin, silk, carboxymethyl cellulose (CMC), chondroitin, collagen, and gelatin.

9. The facial mask of claim 1, wherein further comprising an adhesive border for securing the mask to a subject's face.

10. The facial mask of claim 1, further comprising a plurality of PRPs distributed within said polymeric matrix layer.

11. (canceled)

12. A cosmetic composition, comprising:
a plurality of exosomes derived from human foreskin fibroblasts or an embryonic fibroblast cell line, and
one or more differentiation-inducing factor.

13. The cosmetic composition of claim 12, further comprising one or more differentiation-inducing factors.

14. The cosmetic composition of claim 13, wherein said one or more differentiation-inducing factors comprise any of one or more miRNAs, one or more peptides, one or more growth factors.

15. The cosmetic composition of claim 14, wherein any of said one or more miRNAs, said one or more peptides and said one or more growth factors are extracted from exosomes secreted by said human foreskin fibroblasts or embryonic fibroblast cells.

16. The cosmetic composition of claim 12, further comprising a human foreskin fibroblast or an embryonic cell line culture medium.

17. A facial mask, comprising:
a polymeric matrix having a plurality of protrusions configured for penetrating into stratum corneum of facial skin, and
a cosmetic composition distributed within said polymeric matrix, wherein the cosmetic composition comprises:
a plurality of exosomes derived from human foreskin fibroblasts or an embryonic fibroblast cell line, and
one or more differentiation-inducing factors.

18. The facial mask of claim 17, wherein said cosmetic composition further comprises one or more differentiation-inducing factors.

19.-20. (canceled)
